(19) United States
(12) Patent Application Publication
(10) Pub. No.: US 2004/0138684 A1
(43) Pub. Date: Jul. 15, 2004

(54) Venous anti-reflux implant

(75) Inventor: Darwin Eton, Miami, FL (US)

Correspondence Address: NIXON & VANDERHYE, PC, 1100 N GLEBE ROAD, 8TH FLOOR, ARLINGTON, VA, 22201-4714, US

(73) Assignee: The University of Miami, Miami, FL (US)

(21) Appl. No.: 10/737,754

(22) Filed: Dec. 18, 2003

Related U.S. Application Data
(60) Provisional application No. 60/440,019, filed on Jan. 15, 2003.

(51) Int. Cl. A61B 17/08

(52) U.S. Cl. 606/158

(57) ABSTRACT

An extra-vascular cuff-type implant with inflatable membrane(s) is provided to constrict the vein to serve as a venous valve. The inflatable bladder is inflated by hydraulic pressure from a fluid reservoir that is disposed parallel to the vein to mimic normal physiology.

This application claims the benefit of Provisional Application No. 60/440,019, filed Jan. 15, 2003, the entire contents of which is hereby incorporated by reference in this application.

### BACKGROUND OF THE INVENTION

Venous valves are typically bicuspid valves that are forced together to prevent retrograde flow of blood while permitting forward flow to the heart. The forward flow occurs when the hydrostatic pressure below the valve exceeds the closing pressure of the valve. This happens, for example, when the calf muscle constricts on the venous system (calf muscle pump) or the forward hydrostatic pressure of the pooling venous blood exceeds the weight of blood above the valve, forcing it to open. When an incompetent valve attempts to close in response to a pressure gradient across the valve, however, the valve leaflets do not seal properly such that retrograde flow of blood will occur. When the valve is incompetent and leaks, venous stasis disease develops.

Venous vascular insufficiency results in disabling leg swelling and ulceration in about 1,000,000 Americans annually. The treatment of choice is leg elevation to facilitate blood flow to the heart and high pressure hosiery to provide the required hydrostatic pressure below the incompetent valve(s). Unfortunately, patient compliance for these treatments is poor, resulting in chronic severe disability for those affected.

Other alternatives, such as venous valve surgery, have thus far not proved to be consistently successful over the long term. For example, external vein valve collars of fixed constrictive diameters have been developed to proximate leaky vein valve leaflets. They have not been adopted, however, due to variable outcomes. More specifically, a fixed collar was developed as a rigid snap on device that constricts the vein so that the leaky valve leaflets overlap. Unfortunately, vein symbiosis, scarification, and progressive valvular deterioration have limited their long term benefit.

### BRIEF DESCRIPTION OF THE INVENTION

A need therefore remains for a device to overcome the deficiencies of conventional fixed constrictive devices. The invention provides such a device by providing an implantable compliant collar that advantageously responds to positional changes of the patient in a manner corresponding to normal venous hemodynamics.

More specifically, an inflatable collar is provided that, according to the invention, is comprised of a rigid housing or frame for being applied to the target vein such as by snapping the collar around the vein. The collar of the invention may be applied to, e.g., the femoral vein and/or popliteal vein. Inside the shell of the collar, a flexible membrane assembly is provided for selectively constricting the vein segment within the shell. The inner membrane assembly defines alone or with the rigid outer shell at least one and preferably two or more compartments. At least one of the compartments is in flow communication with a reservoir. The reservoir is configured and disposed so that when the patient is standing erect, media flows from the reservoir into the at least one compartment to fill the same and gently close the vein disposed within the rigid shell segment, thereby preventing reflux toward the foot. The flexible membrane can still act as a one way vein valve because the vein is still able to empty in a forward flow fashion toward the heart when the pressure in the venous circulation below the collar exceeds the pressure of the media behind the flexible membrane. The reservoir is further configured and disposed so that when the patient is supine, the media flows from the at least one compartment to at least partially return to the reservoir, so as to release the force gently closing the vein, such that blood can more freely flow along its return path to the heart.

Thus, the invention may be embodied in a venous anti-reflux device comprising a fluid reservoir and a collar assembly, said collar assembly including a flexible membrane defining at least one compartment in flow communication with said fluid reservoir and a shell for supporting said at least one compartment in surrounding relation to a target vein segment, said media reservoir providing a pressure head of media for filling and distending said flexible membrane when said reservoir is disposed vertically above said collar assembly, thereby to constrict a flow passage through the target vein segment.

The invention may also be embodied in a method for selectively constricting a vein segment for preventing retrograde venous flow comprising providing an implantable device including: a columnar fluid reservoir and a collar assembly, said collar assembly including a flexible membrane defining at least one compartment in flow communication with said fluid reservoir and a shell containing the flexible bladder; and mounting the collar assembly in surrounding relation to a target vein segment so that the columnar reservoir is disposed in parallel to and along a portion of the vein segment downstream, relative to antigrade flow, from the collar assembly whereby when the vein segment is disposed in a generally vertical orientation, said fluid reservoir defines a pressure head of media for filling and distending said flexible bladder, thereby to constrict the target vein segment.

### BRIEF DESCRIPTION OF THE DRAWINGS

These and other objects and advantages of this invention, will be more completely understood and appreciated by careful study of the following more detailed description of the presently preferred exemplary embodiments of the invention taken in conjunction with the accompanying drawings, in which:

FIG. 1 is a schematic perspective view illustrating a cuff according to the invention in an unsecured position ready for application to a target vein segment;

FIG. 2 is a perspective view of an extra-vascular implant embodying the invention disposed in surrounding relation to a target vein segment;

FIG. 3 is a schematic top plan view of the extra-vascular implant of FIG. 2 and with the vein shown in collapsed form within the cuff;

FIG. 4A is a cross-sectional view taken along line IV-IV in FIG. 3 before the flexible membrane has been filled and omitting the vein for clarity;

FIG. 4B is a schematic cross-sectional view taken along line IV-IV of FIG. 3 showing the filled membrane according to a first embodiment of the invention; and

FIG. 4C is a cross-sectional view taken along line IV-IV in FIG. 3 showing the filled membrane according to a second embodiment of the invention and omitting the vein for clarity.

### DETAILED DESCRIPTION OF THE INVENTION

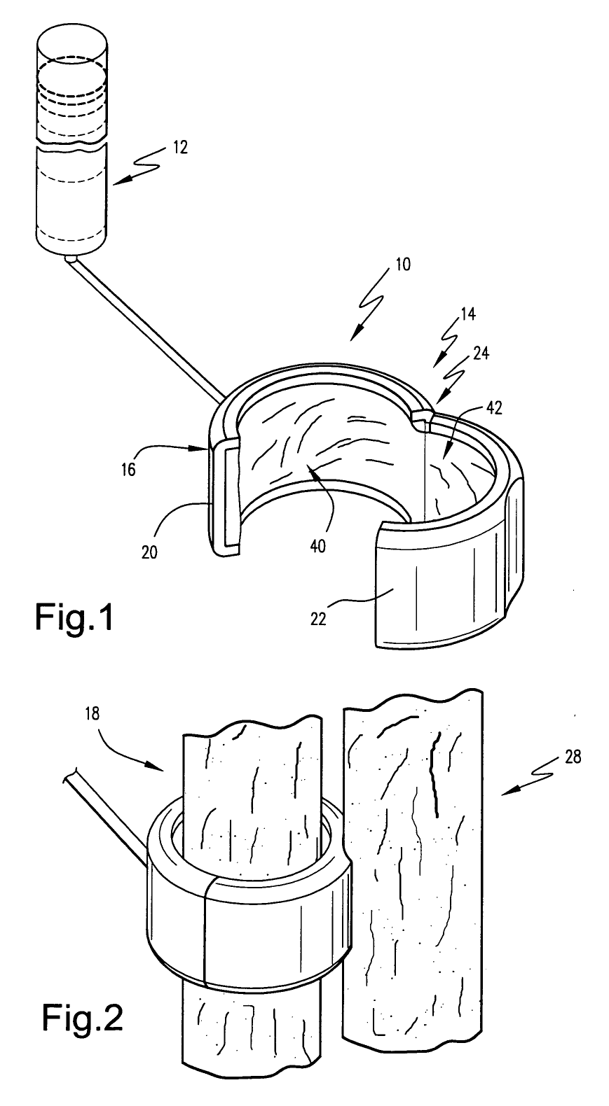

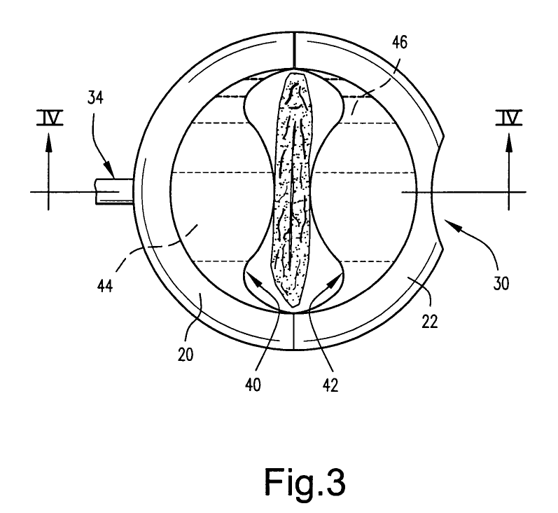

An example of an inflatable collar or cuff device 10 embodying the invention is illustrated in the accompanying FIGURES. In the illustrated example, the device is comprised of a reservoir portion 12 and a collar or cuff portion 14 in fluid flow communication with the reservoir. The collar portion 14 includes a rigid housing or shell 16 which is provided to enable the collar to be fixedly secured in surrounding relation to a target segment 18 of the patient's vein. As illustrated in FIG. 1, in an exemplary embodiment of the invention the shell 16 is formed in two parts 20,22 that are hinged together so that they can be pivoted from an open configuration as shown in FIG. 1 to a closed configuration as shown in FIGS. 2 and 3 to surround the target vein segment 18. As also illustrated in FIG. 2, typically an artery 28 will be disposed in parallel side by side relation to the target vein segment 18. Accordingly, in the illustrated embodiment, one of the shell halves is recessed as at 30 to accommodate the artery.

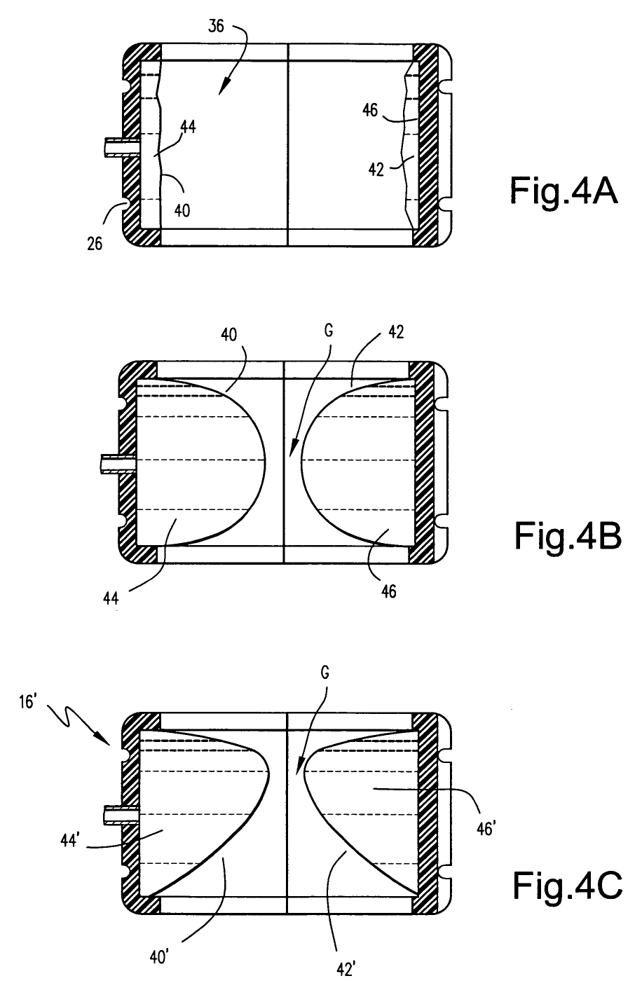

In the illustrated embodiment, the shell parts are hinged at an integral, living hinge 24. When the shell parts are pivoted to the closed configuration of FIGS. 2-3, they are secured together. In an exemplary embodiment, one or more sutures is looped around the shell to hold the shell in its closed disposition. If deemed necessary or desirable, circumferential grooves 26 as schematically shown in FIGS. 4A-4C may be defined about the outer circumference of the shell 16 for receiving and properly positioning the suture material. In the alternative, a protrusion such as a knob or hook may be provided so that the shell halves 20,22 snap and secure together or to receive e.g., suture material. In an exemplary embodiment, the cuff has a height of about 1 cm and an interior diameter of the shell is between about 1.5 and 2 cm to encompass a variety of vein diameters.

Mounted within the housing or shell 16 is an inflatable membrane or bladder assembly for selectively receiving media from the reservoir 12, as described in greater detail hereinbelow. In an exemplary embodiment, as best illustrated in FIGS. 4A-4C, the shell halves 20,22 are generally C-shaped in vertical section to define an inflatable membrane receiving pocket. The inflatable membrane or bladder assembly is preferably comprised of at least two compartments respectively defined by the membrane that are closed but for their communication with each other and with the reservoir 12. In the embodiment illustrated in FIGS. 1 and 3, the inflatable membrane assembly is provided as two inflatable membrane compartments 44,46, communication between the membrane compartments is provided adjacent the hinge 24 and media flows into the inflatable membrane assembly via conduit 32 and port 34 as illustrated e.g., in FIGS. 1 and 4.

As noted above, the membrane assembly may be comprised of inflatable membranes or bladders. In the illustrated embodiment, the fillable compartments of the membrane assembly are defined by membranes suitably secured to the shell halves, so that the fillable compartments are defined between the flexible membranes and the respective wall of the respective shell half. Thus, FIG. 4A schematically illustrates the closed shell with the membrane(s) 40,42 in an uninflated or not fully inflated configuration so that blood is free to flow through the vein segment 18. The membrane is preformed or distendable so that it will transition from the unfilled configuration of FIG. 4A to the fully inflated configuration of FIGS. 3, 4B and/or 4C when media flows from the reservoir to the compartments 44,46;44′,46′. As an alternative to securing a membrane as in the illustrated embodiment, the fillable compartment(s) can be defined by separately formed bladders secured within the shell halves and in flow communication with port 34.

The fillable compartment(s) define a vein receiving opening 36 when the shell 16 is secured closed. However, in the presently preferred embodiment, once the fillable compartments are fully filled and distended, a gap G is still defined between opposing sides of the filled compartments 44,46;44′,46′, as illustrated in FIGS. 3, 4B, and 4C. This gap prevents the membranes 40,42;40′,42′ from compressing the walls of the vein 18 on each other. For example, the opposing membrane walls in a collar with two compartments filled with fluid should preferably not get closer than, e.g., the thickness of two vein walls (2 mm). Since the walls of the filled compartments do not get closer than a 2 mm gap as illustrated, the compartments are preferably inflatable but not expandable so that the compartments define a prescribed shape when fully inflated. In one example, as illustrated in FIG. 4B, the protruding compartments are generally parabolic in vertical section. In an alternate embodiment, as illustrated in FIG. 4C, the closest point between the inflated compartments 44′,46′ is at one vertical end of the shell 16′, e.g., the vertically upper end. While two tillable compartments are provided in the illustrated embodiment, it is to be understood that additional tillable compartments may be provided as deemed necessary or desirable to define the proper inflated bladder profile for occluding the blood vessel when the patient is standing erect.

As noted above, the membrane or bladder compartments are selectively filled with a media that flows thereto from a reservoir 12. The media reservoir has a longitudinal axis that extends substantially perpendicular to a horizontal plane of the collar portion so that it will be disposed vertically above the collar assembly when the patient is standing erect. In an exemplary embodiment, the fluid reservoir is defined as a fluid column disposed generally in parallel to the target vein so as to be exposed to the same gravitational and positional influences as the patient's venous system. The fluid reservoir may be formed from a flexible membrane or, more preferably, as a rigid or semi-rigid structure, as deemed necessary or desirable to provide for a reliable flow connection to the inflatable bladder and to resist compressive external forces which may preclude re-filing of the reservoir when the patient is supine. As presently proposed, the fluid reservoir would be implanted in continuity with the collar 12 in the subcontaneous space. The size of the column is defined by, e.g., the density of the fluid within it and by the weight of the venous blood between the last competent valve and the collar implant site. The latter may be calculated invasively from duplex scan data. Also, implantation of two or more devices in series would shorten the length of each column. This is because the reservoir size dictates how much retrograde flow pressure can be resistant by the device and multiple devices will reduce the amount of retrograde flow each device is designed to resist.

The media contained within the reservoir may be either a fluid, a solid or a combination thereof. In a presently preferred embodiment, the media provided in the reservoir is a biocompatible fluid. An exemplary fluid is saline which would be provided to mimic a column of blood. In such embodiment, a relatively large/long fluid reservoir would be required to mimic the pressure of a corresponding column of blood. In general, suitable fluids will have a low viscosity and high density to provide appropriate flowability and pressure as the patient's position changes. It will be appreciated that a relatively limited flow of fluid from the reservoir to the inflatable membrane in conjunction with the pressure head created by the column of fluid will be sufficient to advantageously allow the venous segment to resist retrograde flow while allowing the desired anti-flux flow.

As noted above, the media contained in the reservoir may be a solid, such as flowable particulate matter. As a further alternative, a solid and a fluid can be paired, the fluid providing the flowability for compartment filling and displacement of the membranes, and the solid providing the appropriate force (pressure) to displace the fluid. In such a configuration, the weight (solid) would act as a force generator and the fluid as the vein closure actuator.

The reservoir 12 itself is preferably provided as an externally supported graft disposed in the subcutaneous tissue and fluidly coupled to the cuff, as mentioned above. By way of example, the reservoir may be formed from PTFE so that the flow of media from the reservoir to the compartments of the collar is due to the patient's position rather than external compressive forces on the reservoir. The media connection from the reservoir to the cuff may be a 5 mm diameter conduit 32 that is e.g., friction fit to a port 34 defined in the wall of the collar shell half 20. A suture may be secured around the friction fit components to enhance the security of the connection.

As is apparent, the proposed constricting collar of the invention does not sit over the existing incompetent vein valve, can accommodate changes in pressure and volume, and does not have to be configured to precisely fit the vein segment since the only absolute dimension is the distance between opposing sides of the deformable membranes within the rigid shell. This dimension prevents the membrane from compressing and crushing the walls of the vein on each other.

The inflatable cuff of the invention advantageously mimics the venous circulation by defining a parallel circuit that responds to gravity just as the venous circulation does but prevents deleterious reverse flow which causes venous hypertension and skin ulceration at the ankles. The device is provided wholly outside the blood stream so that the endothelial layer within the vein is not disrupted. The device of the invention is also easy to implant, requiring little time to implant and requiring only the skill of a general surgeon. In this regard, it is noted that long graft tunnels are routinely created for routing arterial grafts. The same equipment may be used for tunneling to accommodate the columnar reservoir of the invention.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A venous anti-reflux device comprising:
a fluid reservoir and a collar assembly, said collar assembly including a flexible membrane defining at least one compartment in flow communication with said fluid reservoir and a housing for supporting said at least one compartment in surrounding relation to a target vein segment, said media reservoir providing a pressure head of media for filling and distending said flexible membrane when said reservoir is disposed vertically above said collar assembly, thereby to constrict a flow passage through the target vein segment.

2. A venous anti-reflux device as in claim 1, wherein when said reservoir is disposed laterally adjacent or below said collar, said media is substantially free to flow from said compartment toward said reservoir.

3. A venous anti-reflux device as in claim 1, wherein when said reservoir is an elongate, columnar reservoir having a longitudinal axis generally perpendicular to a horizontal plane of said collar.

4. A venous anti-reflux device as in claim 1, wherein the housing is formed in first and second parts that are hingedly coupled together so as to be pivotable from an open configuration to a closed configuration to surround the target vein segment.

5. A venous anti-reflux device as in claim 4, wherein one of said parts is recessed to accommodate an artery adjacent the target vein segment.

6. A venous anti-reflux device as in claim 4, wherein said parts are hinged at an integral, living hinge.

7. A venous anti-reflux device as in claim 4, further comprising at least one suture loop around the housing for holding said parts in said closed configuration.

8. A venous anti-reflux device as in claim 7, further comprising a circumferential groove defined about the outer circumference of the housing for receiving said at least one suture loop

9. A venous anti-reflux device as in claim 1, wherein said flexible membrane defines two diametrically opposed inflatable membrane compartments.

10. A venous anti-reflux device as in claim 1, wherein said flow communication between said flexible membrane and said fluid reservoir comprises a conduit defined between said reservoir and a port of said housing.

11. A venous anti-reflux device as in claim 1, wherein said flexible membrane is constructed and arranged so that the filled and distended membrane reduces a dimension of a passage through the collar assembly to not less than 2 mm whereby walls of the target vein segment collapsed but not crushed therein.

12. A venous anti-reflux device as in claim 1, wherein the media contained in the reservoir comprises a liquid.

13. A venous anti-reflux device as in claim 12, wherein the media in the reservoir comprises saline.

14. A method for selectively constricting a vein segment for preventing retrograde venous flow comprising:
providing an implantable device including: a columnar fluid reservoir and a collar assembly, said collar assembly including a flexible membrane defining at least one compartment in flow communication with said fluid reservoir and a housing engaging the flexible membrane;
mounting the collar assembly in surrounding relation to a target vein segment so that the columnar reservoir is disposed in parallel to and along a portion of the vein segment downstream, relative to antigrade flow, from the collar assembly whereby when the vein segment is disposed in a generally vertical orientation, said fluid reservoir defines a pressure head of media for filling and distending said flexible bladder, thereby to constrict the target vein segment.

15. A method as in claim 14, wherein when the vein segment is disposed in a generally horizontal orientation media is free to flow from said flexible membrane toward said reservoir.

16. A method as in claim 14, wherein the housing is formed in first and second parts that are hingedly coupled together and wherein said mounting comprises pivoting said parts of said housing from an open configuration to a closed configuration surrounding said target vein segments.

17. A method as in claim 16, further comprising securing said parts in said closed configuration.

18. A method as in claim 17, wherein said securing comprises looping suture material around the housing for holding said parts in said closed configuration.
